(19) United States
(12) Patent Application Publication
(10) Pub. No.: US 2017/0340547 A1
(43) Pub. Date: Nov. 30, 2017

(54) AGENTS AND METHODS FOR THE TEMPORARY SHAPING OF KERATIN-CONTAINING FIBERS

(71) Applicant: HENKEL AG & CO. KGAA, Duesseldorf (DE)

(72) Inventors: Thomas FOERSTER, Duesseldorf (DE); Rolf BAYERSDOERFER, Hamburg (DE); Thorsten KNAPPE, Schenefeld (DE)

(73) Assignee: Henkel AG & Co. KGaA, Duesseldorf (DE)

(21) Appl. No.: 15/534,222

(22) PCT Filed: Nov. 2, 2015
(86) PCT No.: PCT/EP2015/075395

§ 371 Date: Jun. 8, 2017

(30) Foreign Application Priority Data
Dec. 10, 2014 (DE) 10 2014 225 430.2

(51) Int. Cl. A61K 8/81; A45D 34/04; A45D 7/04; A61K 8/34; B05B 9/00; A61Q 5/06

(52) U.S. Cl. CPC A61K 8/8147; B05B 9/005; B05B 9/002; A61Q 5/06; A45D 7/04; A61K 8/34; A45D 34/04; A61K 2800/87

(57) ABSTRACT

The disclosure relates to a cosmetic product, comprising a) a cosmetic preparation, containing, in relation to its total weight, a1) 60 to 98 wt. % of at least one polar solvent; a2) 0.1 to 20 wt. % of at least one copolymer from i) styrene ii) methacrylic acid iii) and optionally other monomers; b) a device for flash evaporation of the cosmetic preparation a), and to a method using corresponding products and to the use of the cosmetic preparation a) as process material in a device for flash evaporation.

### CROSS-REFERENCE TO RELATED APPLICATION

This application is a U.S. National-Stage entry under 35 U.S.C. §371 based on International Application No. PCT/EP2015/075395, filed Nov. 2, 2015, which was published under PCT Article 21(2) and which claims priority to German Application No. 10 2014 225 430.2, filed Dec. 10, 2014, which are all hereby incorporated in their entirety by reference.

### TECHNICAL FIELD

The application relates to the technical field of temporarily deforming keratin-containing fibers, especially human hair. The subject matter of the application are specific cosmetic formulations for hair, which are suitable for application on keratin-containing fibers by employing a flash evaporation process. Moreover, the use of these cosmetic formulations for hair in devices for flash evaporation and methods for the temporary deformation of keratin-containing fibers are the subject matter of the present application.

### BACKGROUND

An attractively appearing hairstyle is generally regarded at the present time as an indispensable part of a groomed appearance. Moreover, on the basis of current fashion trends, hairstyles appear time and again as chic, which can be built up for many types of hair only with the use of consolidating active ingredients or maintained for a longer period of up to several days. Hair-treating agents, which are used for the temporary or permanent shaping of hair, therefore play an important role. Whereas the chemical structure of the keratin-containing fibers is modified by reduction and oxidation during permanent transformations, such modifications of the chemical structure do not take place during the temporary transformation. Appropriate materials for temporary deformations usually contain synthetic polymers and/or waxes as the consolidating active ingredient.

The most important property of a material for the temporary deformation of keratin-containing fibers, also named styling material in the following, is to provide the treated fibers in the newly modeled form, that is, a form impressed on the fibers, with the best possible support. If the keratin-containing fibers are human hair, it is customary to speak also of long-lasting styling. The duration of the styling is determined essentially by the nature and amount of the active, consolidating material used; however, the further components of the styling material and the form of application may also have an effect.

In the area of temporary deformation of keratin-containing fibers, especially the spray application of appropriate cosmetic preparations is of great importance, these preparations usually being applied as pump sprays or as aerosol sprays. For this purpose, the cosmetic preparations are packaged in a dispensing device, from which they are sprayed either through mechanical action or with the help of a propellant and a valve. Both methods have obvious disadvantages. Whereas pump sprays generally are not suitable for a long-lasting uniform spray applications of cosmetic preparations for the hair, aerosol sprays are based on the use of propellants or propellant gases which, on the one hand, do not develop a cosmetic effect and, on the other, can pose a risk to the consumer when handled improperly.

Against this background, there is a need for alternative routes for atomizing cosmetic preparations for the hair. Flash evaporation has proven to be such an alternative spraying method. For this method, which is described, for example, in the international patent application WO 2001/83071 A1 (Henkel), a liquid or pasty solvent-containing composition is heated in a closed space to a temperature, which is above the boiling point of the solvent, as a result of which pressure is produced in the composition. Upon relieving (throttling) the pressure, the liquid vaporizes and can subsequently be atomized, for example, by employing a suitable nozzle.

Even though flash evaporation is suitable, in principle, for the spray application of cosmetic preparations for the hair, at the same time, not every cosmetic preparation for the hair can be atomized by a flash evaporation method. This is due, on the one hand, to the heating of the cosmetic preparation, which is necessary for the flash evaporation, and, on the other, to the specific properties of the spray mist produced by the flash evaporation, for example, the droplet size and droplet density in the spray mist produced.

### BRIEF SUMMARY

A cosmetic product is provided herein. The cosmetic product includes a cosmetic preparation. The cosmetic preparation includes, based on its total weight, a1) about 60 to about 98% by weight of at least one polar solvent. The cosmetic preparation further includes, based on its total weight, a2) about 0.1 to about 20% by weight of at least one copolymer of i) styrene, ii) methacrylic acid, and iii) optionally, further monomers. The cosmetic product further includes a device for the flash evaporation of the cosmetic preparation a).

A cosmetic preparation is also provided herein. The cosmetic preparation includes, based on its total weight, a1) about 60 to about 98% by weight of at least one polar solvent. The cosmetic preparation further includes, based on its total weight, a2) about 0.1 to about 20% by weight of at least one copolymer of i) styrene, ii) methacrylic acid, and iii) optionally, further monomers. The cosmetic product further includes a device for the flash evaporation of the cosmetic preparation a). The cosmetic preparation is utilized as process materials in a device for flash evaporation.

A method for the temporary deformation of keratin-containing fibers, the method includes impacting upon the keratin-containing fibers utilizing a device for flash evaporation with a cosmetic preparation including, based on its total weight, a1) about 60 to about 98% by weight of at least one polar solvent. The cosmetic preparation further includes, based on its total weight, a2) about 0.1 to about 20% by weight of at least one copolymer of i) styrene, ii) methacrylic acid, and iii) optionally, further monomers.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It was therefore an object of the present disclosure to provide specific cosmetic preparations for hair for temporarily deforming keratin-containing fibers, which, owing to their chemical and physical properties, are suitable for a purpose-directed spray application by employing a device for flash evaporation. Furthermore, the preparations should be suitable for realizing a high degree of holding the hair in place, especially of doing so for a long time, and a high volume effect after an application by employing a flash method. It has turned out that, especially solvent-containing preparations based on a copolymer of styrene and methacrylic acid from the plurality of known, effective cosmetic polymer preparations for the hair, are suitable for solving this task.

A first object of the present disclosure accordingly is a cosmetic product, comprising

a) a cosmetic preparation containing, based on the total weight,

a1) about 60 to about 98% by weight of at least one polar solvent;

a2) about 0.1 to about 20% by weight of at least one copolymer of
i) styrene
ii) methacrylic acid
iii) as well as optionally further monomers
   b) a device for the flash evaporation of the cosmetic preparation a).

Preferably, the cosmetic preparation a) is a liquid. The cosmetic preparation a) may also be present as a solution or dispersion, for example, as an emulsion or suspension. Preferred cosmetic preparations a) are present in the form of a solution or a suspension.

As a first significant component, the inventive cosmetic preparation contained about 60 to about 98% by weight of at least one polar solvent a1. Preferred cosmetic products are characterized in that the proportion by weight of the polar solvent a1) amounts to about 70 to about 98% by weight and preferably about 80 to about 98% by weight of the total weight of the cosmetic preparation a). Appropriate materials are distinguished by a good cosmetic effect and, at the same time, by good applicability.

In order to improve the application properties of the inventive cosmetic preparations while at the same time minimizing the thermal strain on any active ingredients or additives in the course of the flash evaporation process, it has proven to be advantageous to use polar solvents a1), which have a boiling point (20° C., 1013 mbar) of between about 50 and about 110° C. and preferably of between about 70 and about 105° C. In this connection, ethanol, isopropanol and water have proven to be particularly suitable and are preferred as polar solvent a1) for this reason.

Polar solvents a1) or solvent systems, which are particularly preferred, are

polar solvents a1), comprising more than about 70% by weight, preferably more than about 80% by weight and particularly more than about 90% by weight of ethanol, in each case based on the total weight of the polar solvent;

polar solvents a1), comprising more than about 80% by weight, preferably more than 90% by weight and particularly more than about 95% by weight, based on the total weight of the polar solvent, of a mixture of ethanol and water, the ratio by weight of ethanol to water preferably being about 10:1 to about 3:1 and, particularly, about 8:1 to about 4:1;

polar solvents a1), comprising more than about 80% by weight, preferably more than about 88% by weight and particularly more than about 92% by weight of water, in each case based on the total weight of the polar solvent.

Cosmetic preparations, which are particularly preferred, are characterized in that the proportion of volatile compounds does not exceed about 55% by weight. The group of these volatile components includes, inter alia, also the polar solvent ethanol. Accordingly, in other words, particularly preferred cosmetic preparations are characterized in that the ratio by weight of ethanol to the total weight of the cosmetic preparation does not exceed about 55% by weight, preferably is about 10 to about 55% by weight, particularly about 25 to about 55% by weight and especially about 40 to about 55% by weight.

A further preferred object of the present disclosure accordingly is a cosmetic product, comprising

a) a cosmetic preparation containing, based on its total weight,

a1) about 60 to about 98% by weight of at least one polar solvent, wherein the ratio of the weight ethanol to the total weight of the cosmetic preparation does not exceed about 55% by weight, preferably is between about 10 and about 55% by weight, particularly about 25 to about 55% by weight and especially about 40 to about 55% by weight;

a2) about 0.1 to about 20% by weight of at least one copolymer from the following monomers
i) styrene
ii) methacrylic acid
iii) as well as, optionally, further monomers
   b) a device for the flash evaporation of the cosmetic preparation a), comprising

b1) a container b1), which can be closed off and opened up with a valve

b2) a heating device, which enables a cosmetic preparation in the container b1) to be heated and

b3) and a nozzle b3), which enables the cosmetic preparation a) to be atomized.

A preferred combination of polar solvents a1) comprises water and ethanol, wherein

the ratio by weight of water and ethanol to the total weight of the polar solvent a1) preferably is at least about 60% by weight, especially at least about 80% by weight, particularly at least about 90% by weight and, more particularly, at least about 95% by weight and, furthermore preferably,

the ratio by weight of water to ethanol is about 5:1 to about 1:5, preferably about 2:1 to about 1:2 and particularly about 5:4 to about 4:5.

A further preferred object of the present disclosure accordingly is a cosmetic product, comprising

a) a cosmetic preparation containing, based on its total weight,

a1) about 60 to about 98% by weight of at least one polar solvent, wherein
the ratio by weight of water and ethanol to the total weight of the polar solvent a1) preferably is at least about 60% by weight, especially at least about 80% by weight, particularly at least about 90% by weight and, more particularly, at least about 95% by weight and
the ratio by weight of water to ethanol is about 5:1 to about 1:5, preferably about 2:1 to about 1:2 and particularly about 5:4 to about 4:5.

a2) about 0.1 to about 20% by weight of at least one copolymer from the following monomers
i) styrene
ii) methacrylic acid
iii) as well as optionally further monomers
   b) a device for the flash evaporation of the cosmetic preparation a), comprising

b1) a container b1), which can be closed off and opened up with a valve

b2) a heating device, which enables a cosmetic preparation in the container b1) to be heated and

b3) a nozzle b3), which enables the cosmetic preparation a) to be atomized.

A second significant component of the inventive cosmetic compositions is copolymer a2). In relation to the ease of production, applicability and cosmetic effect of the inventive cosmetic composition, it has proven to be advantageous if the proportion by weight of copolymer a2) to the total weight of the cosmetic preparation a) is about 0.5 to about 15% by weight and preferably about 1.0 to about 10% by weight.

Copolymer a2) is attributable to the monomers, i) styrene and ii) methacrylic acid, as well as, optionally, to further monomers.

Preferred copolymers a2) preferably comprise at least about 90% by weight, especially at least about 95% by weight and particularly at least about 97% by weight of the monomers i) styrene and ii) methacrylic acid. Particularly preferred copolymers e) are obtained exclusively from the monomers i) styrene and ii) methacrylic acid.

The above-described copolymers a2) are sold, for example, under the name Acudyne® Shine (INCI name: styrene/acrylate copolymer; CAS number 9010-92-8 by Dow Chemicals.

An especially preferred object of the present disclosure accordingly is a cosmetic product, comprising

a) a cosmetic preparation containing, based on the total weight,

a1) about 80 to about 98% by weight of at least one polar solvent;

a2) about 1.0 to about 20% by weight of at least one copolymer of
i) styrene
ii) methacrylic acid
iii) as well as optionally further monomers
   b) a device for the flash evaporation of the cosmetic preparation a).

The copolymer a2) preferably is used in a partly neutralized or neutralized form in the cosmetic materials. Preferably, at least one alkanolamine is used for the neutralization. The alkanolamines, which can be used as the inventive alkalization agent, preferably are selected from primary amines with a C2-C6 basic alkyl, which contains at least one hydroxyl group. Particularly preferred alkanolamines are selected from the group comprising 2-amino-1-ethanol (mono ethanol amine), tris-(2-hydroxyethyl)-amine, (tri ethanol amine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol. Alkanolamines, particularly preferred as contemplated herein, are selected from the group comprising 2-amino-1-ethanol, 2-amino-2-methy-1-propanol and 2-amino-2-methyl-1,3-propanediol. 2-Amino-2-methylpropanol has proven to be a particularly suitable neutralizing agent. Cosmetic materials, which are preferred as contemplated herein, contain at least one alkanolamine, preferably 2-amino-2-methylpropanol. The 2-amino-2-methylpropanol is used in the inventive materials preferably in an amount, which does not exceed the amount required for neutralizing the copolymer a2). Preferably, the amount of 2-amino-2-methylpropanol, used in the inventive materials, is 80 to 100%, preferably 90 to 100% and particularly 95 to 100% of the amount required for the complete neutralization of the copolymer a2). In a preferred embodiment, the proportion by weight of the 2-amino-2-methylpropanol in the cosmetic preparation a) is about 0.1 to about 4.0% by weight, preferably about 0.5 to about 3.0% by weight and particularly about 1.0 to about 2.0% by weight.

Aside from the cosmetic preparation a), the inventive cosmetic products comprise furthermore a device for flash evaporation. Within the scope of the present disclosure, the expression “flash evaporation” refers to the formation of vapor when the pressure is lowered in a closed space, which is filled with a liquid and is under pressure in comparison to the surroundings. An appropriate pressure can be produced, for example, in that an amount of cosmetic preparation a) is heated in a closed space to a temperature T1. In the closed space, the liquid has a saturation pressure p1 at a given temperature T1. If the closed space is opened, for example, by employing a valve to a relaxation space, which is not under pressure with the pressure p0<p1, the pressure in the previously closed space drops and, as the new pressure level spreads, the cosmetic preparation a) or the solvent or parts of the solvent, contained in the cosmetic preparation, are evaporated. The resulting vapor or spray mist formed can be used for the application of specific cosmetic preparations.

If, starting out from standard conditions (T0=25° C., p0=1,000 bar), the cosmetic preparation a) is heated in a closed space, aside from an elevated temperature, an elevated pressure of the cosmetic preparation a) results. This elevated pressure can be relieved in a relaxation space to a pressure p0, for example, to the pressure of the surrounding area (p0=1,000 bar), as a result of which partial evaporation of the cosmetic preparation a) is attained.

The pressure on the cosmetic preparation a) can be relieved directly in the space, in which the cosmetic preparation a) was previously heated. Alternatively, the cosmetic preparation a), which is heated and under pressure, can also be transported into a second space, in which the pressure is then relieved subsequently.

In other words, flash evaporation is a method, for which the cosmetic preparation a) is heated in a closed container by a heater to temperatures above the surrounding temperature; a pressure above the surrounding temperature results in the container and the pressure on the heated cosmetic preparation a) is subsequently relieved out of the container into the surroundings.

Accordingly, a device for flash evaporation is a device, which comprises a container and a heater and is equipped in such a manner, that a cosmetic preparation a) in the closed container can be heated by employing the heater to temperatures above the surrounding temperature in such a way, that a pressure above the surrounding pressure develops in the container and the pressure on the heated cosmetic preparation a) can be relieved from the container into the surroundings.

Simultaneously with or after the pressure is relieved, the cosmetic preparation a) can be supplied to a nozzle, such that the properties, for example, of the vapor or spray mist, produced by the flash evaporation, especially the droplet size or the droplet density, but also the spray width and the shape of the spray cone can be affected. The use of nozzles, preferably atomizing nozzles, therefore is preferred. The specific type or configuration of the nozzle can be established selectively, depending on the properties of the respective spray mist.

To summarize, a preferred device for the flash evaporation has
b1) a container b1), which can be opened and closed by employing a valve and which defines a closed inner space, in which the cosmetic preparation can be accommodated,
b2) a heating device b2), which enables a cosmetic preparation in the container b1) to be heated.

The use of an additional nozzle b3), which enables the cosmetic preparation a), escaping from the container, to be atomized is especially preferred. Alternatively to a valve, a closing element, which has a comparable action and closes off or releases an associated opening in the container by an appropriate positional change, can also be used.

A further preferred object of the present disclosure accordingly is a cosmetic product, comprising
a) a cosmetic preparation containing, based on the total weight,

a1) about 60 to about 98% by weight of at least one polar solvent;

a2) about 0.1 to about 20% by weight of at least one copolymer of
i) styrene
ii) methacrylic acid
iii) as well as, optionally, further monomers;
b) a device for the flash evaporation of the cosmetic preparation a), the device for the flash evaporation comprising a container b1) and a heater b2) and being configured in such a manner that
   the cosmetic preparation a) can be accommodated in the interior of the container b1)
   the interior of the container b1), which is at least partially filled with the cosmetic preparation a), can be closed off,
   the cosmetic preparation a) in the closed interior of the container b1) can be heated with the heater b2) while the pressure increases.

A further preferred object of the present disclosure accordingly is a cosmetic product, comprising
a) a cosmetic preparation containing, based on the total weight,

a1) about 60 to about 98% by weight of at least one polar solvent;

a2) about 0.1 to about 20% by weight of at least one copolymer of
i) styrene
ii) methacrylic acid
iii) as well as optionally further monomers
b) a device for the flash evaporation of the cosmetic preparation a), comprising

b1) a container b1), which can be closed off and opened up with a valve

b2) a heating device, which enables a cosmetic preparation in the container b1) to be heated and

b3) a nozzle b3), which enables the cosmetic preparation a) to be atomized.

In other words, a particularly preferred object of the present disclosure is a cosmetic product, comprising
a) a cosmetic preparation containing, based on its total weight,

a1) about 60 to about 98% by weight of at least one polar solvent;

a2) about 0.1 to about 20% by weight of at least one copolymer from the following monomers
i) styrene
ii) methacrylic acid
iii) as well as, optionally, further monomers
b) a device for the flash evaporation of the cosmetic preparation a), the device for the flash evaporation comprising a container b1) and a heater b2) and being configured in such a manner that
   the cosmetic preparation a) can be accommodated in the interior of the container b1)
   the interior of the container b1), which is at least partially filled with the cosmetic preparation a), can be closed off,
   the cosmetic preparation a) in the closed interior of the container b1) can be heated with the heater b2) while the pressure increases,
   the pressure on the heated cosmetic preparation a) from the interior of the container b1) can be lowered by being relieved into the surroundings.

The container b1), in which the cosmetic preparation is heated, is configured in such a way that it enables this container to be closed off completely from the environment while the cosmetic preparation a) is being heated and, after the heating, to be opened up to enable the cosmetic preparation a) to be flash evaporated. This may be ensured, for example, by a component for controlling flow, especially by a valve.

Preferably, the container b1), in which the cosmetic preparation is heated, is in contact with a further container, from which the amount of cosmetic preparation, which is to be flash evaporated, is transferred before being heated in container b1). The access between this reservoir and the container b1) can therefore be opened and closed by an appropriate device, such as a valve. This further container preferably is in the form of a reservoir, that is, it preferably comprises a multiple, such as more than about 10× and preferably more than about 50× of the amount of cosmetic preparation necessary for one evaporation process. In other words, the capacity of the further container/reservoir preferably is a multiple, for example, more than about 10 times, preferably more than about 20 times and particularly more than about 50 times the capacity of the container b1).

A further, particularly preferred object of the present disclosure therefore is a cosmetic product, comprising
a) a cosmetic preparation containing, based on its total weight,

a1) about 60 to about 98% by weight of at least one polar solvent;

a2) about 0.1 to about 20% by weight of at least one copolymer from the following monomers
i) styrene
ii) methacrylic acid
iii) as well as, optionally, further monomers
b) a device for the flash evaporation of the cosmetic preparation a), comprising
b1) a container b1), which can be closed off and opened up with a valve
b2) a heating device, which enables a cosmetic preparation in the closed container b1) to be heated and
b3) a nozzle b3), which enables the cosmetic preparation a) to be atomized.
c) and a reservoir for the cosmetic preparation a), from which the cosmetic preparation a) can reach the container b1),
   wherein the access between the reservoir and the container b1) has a flow-regulating component, such that the flow of the cosmetic preparation a) from the reservoir into the container b1) can be interrupted;
   the capacity of the reservoir is at least about 10 fold, preferably at least about 20 fold and particularly at least about 50 fold that of the container b1).

The reservoir is not a pressure container and the cosmetic preparation in the reservoir is not under pressure; in other words, the pressure in the interior of the reservoir corresponds to atmospheric pressure (air pressure or atmospheric pressure). Appropriate cosmetic products do not contain, for example, a propellant. The cosmetic product also does not have a pump device, which is suitable for releasing or spraying the cosmetic preparation into the environment without the action of a device for flash evaporation.

A particularly preferred object of the present disclosure accordingly is a cosmetic product, comprising
a) a cosmetic preparation containing, based on its total weight,

a1) about 60 to about 98% by weight of at least one polar solvent;

a2) about 0.1 to about 20% by weight of at least one copolymer from the following monomers
i) styrene
ii) methacrylic acid
iii) as well as, optionally, further monomers
b) a device for the flash evaporation of the cosmetic preparation a), comprising

b1) a container b1), which can be closed off and opened up with a valve

b2) a heating device, which enables a cosmetic preparation in the closed container b1) to be heated,

b3) a nozzle b3), which enables the cosmetic preparation a) to be atomized and
c) a reservoir for the cosmetic preparation a), from which the cosmetic preparation a) can reach the container b1),
   wherein the access between the reservoir and the container b1) has a flow-regulating component, such that the flow of the cosmetic preparation a) from the reservoir into the container b1) can be interrupted;
   the capacity of the reservoir is at least about 10 fold and preferably at least about 50 fold that of the container b1);
   the pressure in the interior of the reservoir corresponds to that of the surroundings.

An especially preferred object of the present disclosure accordingly is a cosmetic product, comprising
a) a cosmetic preparation containing, based on its total weight,

a1) about 60 to about 98% by weight of at least one polar solvent;

a2) about 0.1 to about 20% by weight of at least one copolymer of
i) styrene
ii) methacrylic acid
iii) as well as, optionally, further monomers
b) a device for the flash evaporation of the cosmetic preparation a), comprising

b1) a container b1), which can be closed off and opened up with a valve

b2) a heating device, which enables a cosmetic preparation in the closed container b1) to be heated

b3) and a nozzle b3), which enables the cosmetic preparation a) to be atomized.
c) and a reservoir for the cosmetic preparation a), from which the cosmetic preparation a) can reach the container b1),
   wherein the access between the reservoir and the container b1) has a flow-regulating component, such that the flow of the cosmetic preparation a) from the reservoir into the container b1) can be interrupted;
   the capacity of the reservoir is at least about 10 fold and preferably at least about 50 fold that of the container b1);
   the pressure in the interior of the reservoir corresponds to the surrounding pressure and the cosmetic product does not comprise a propellant.

Furthermore, cosmetic products are preferred, which comprise
a) a cosmetic preparation containing, based on its total weight,

a1) about 60 to about 98% by weight of at least one polar solvent;

a2) about 0.1 to about 20% by weight of at least one copolymer of
i) styrene
ii) methacrylic acid
iii) as well as, optionally, further monomers
b) a device for the flash evaporation of the cosmetic preparation a), comprising
b1) a container b1), which can be closed off and opened up with a valve b2) a heating device, which enables a cosmetic preparation in the closed container b1) to be heated
b3) and a nozzle b3), which enables the cosmetic preparation a) to be atomized.
c) and a reservoir for the cosmetic preparation a), from which the cosmetic preparation a) can reach the container b1),
   wherein the access between the reservoir and the container b1) has a flow-regulating component, such that the flow of the cosmetic preparation a) from the reservoir into the container b1) can be interrupted;
   the capacity of the reservoir is at least about 10 fold and preferably at least about 50 fold that of the container b1);
   the pressure in the interior of the reservoir corresponds to atmospheric pressure,
      the cosmetic product not having a pump device, which is suitable for releasing or spraying the cosmetic preparation a) without the action of a device for flash evaporation.

To summarize, a particularly preferred object of the present disclosure accordingly is a cosmetic product, comprising
a) a cosmetic preparation containing, based on its total weight,

a1) about 60 to about 98% by weight of at least one polar solvent;

a2) about 0.1 to about 20% by weight of at least one copolymer of
i) styrene
ii) methacrylic acid
iii) as well as, optionally, further monomers
b) a device for the flash evaporation of the cosmetic preparation a), comprising

b1) a container b1), which can be closed off and opened up with a valve

b2) a heating device, which enables a cosmetic preparation in the closed container b1) to be heated

b3) and a nozzle b3), which enables the cosmetic preparation a) to be atomized.

c) and a reservoir for the cosmetic preparation a), from which the cosmetic preparation a) can reach the container b1),
wherein the access between the reservoir and the container b1) has a flow-regulating component, such that the flow of the cosmetic preparation a) from the reservoir into the container b1) can be interrupted;
the capacity of the reservoir is at least about 10 fold and preferably at least about 50 fold to that of the container b1);
the pressure in the interior of the reservoir corresponds to the atmospheric pressure and the cosmetic product does not comprise a propellant,
   the cosmetic product does not have a pump device, which is suitable for releasing or spraying the cosmetic preparation a) without the action of a device for flash evaporation.

Aside from the two previously described components a1) and a2), the inventive cosmetic preparations a) may contain further active ingredients or additives, especially those active ingredients or additives being preferred, which improve the ease of production, applicability and cosmetic effect of the inventive cosmetic preparations.

An example of a preferred, further active ingredient and additive are the film-forming polymers a3), the use of which in the inventive cosmetic materials is especially preferred.

Permanently as well as temporary cationic, anionic, non-ionic or amphoteric polymers are suitable as film-forming polymers a3). These film-forming polymers may be of synthetic or natural origin. Based on the total weight, preferred cosmetic preparations a) contain about 0.1 to about 20% by weight, preferably about 0.5 to about 15% by weight and particularly about 1.0 to about 10% by weight of a film-forming polymer a3), which is different from polymer a2).

Acrylamide/ammonium acrylate copolymer, acrylamides/DMAPA acrylates/methoxy PEG methacrylate copolymers are examples of conventional, film-forming polymers a3). Other examples includes acrylamido-propyltrimonium chloride/acrylamide copolymers, acrylamidopropyltrimonium chloride/acrylate copolymers, acrylates/acetoacetoxyethyl methacrylate copolymers, acrylates/acrylamide copolymers, acrylate/ammonium methacrylate copolymers, acrylate/t-butyl acrylamide copolymers acrylate copolymers, acrylate/C1-2 succinates/hydroxyacrylate copolymer, acrylate/lauryl acrylate/stearyl acrylate/ethylamine oxide methacrylate Copolymers, acrylate/octylacrylamide copolymers, acrylate/octyl acrylamide/diphenyl ammodimethicone copolymers, acrylates/stearyl acrylate/ethylamine oxide methacrylate copolymers, acrylate/VA copolymer, acrylate/VP copolymer, adipic acid/diethylenetriamine copolymer, adipic acid/dimethylaminohydroxypropyl diethylenetriamine copolymers, adipic acid/epoxypropyl diethylenetriamine copolymers, adipic acid/lsophthalic acid/neopentyl glycol/trimethylolpropane copolymers, allyl stearate/VA copolymers, amino ethyl acrylate phosphate/acrylate copolymers, amino ethyl propanediol-acrylate/acrylamidecopolymers, aminoethylpropanediol-AMPD-acrylate/diacetoneacrylamide copolymer, ammonium VA/acrylate copolymers, AMPD-acrylates/diacetoneacrylami decopolymers, AMP-acrylate/allyl methacrylate copolymers, AMP-Acrylate/C1-18 alkyl acrylates/C1-8 alkyl acrylamide copolymers, AMP-acrylates/diacetoneacrylamide copolymers, AMP-acrylates/dimethylaminoethyl methacrylate copolymers, Bacillus/rice bran extract/soybean extract ferment filtrates, bis-butyloxyamodimethicone/PEG-60 copolymers, butyl acrylate/ethylhexyl methacrylate copolymers, butyl acrylate/hydroxypropyl dimethicone acrylate copolymers, butylated PVP, butyl ester of ethylene/MA copolymers, butyl ester of PVM/MA copolymers, calcium/sodium PVM/MA copolymers, corn starch/acrylamide/sodium acrylate copolymers, diethylene glycolamine/epichlorohydrin/piperazine copolymers, dimethicone crosspolymer, diphenyl amodimethicone, ethyl ester of PVM/MA copolymer, hydrolyzed wheat protein/PVP crosspolymers, isobutylene/ethyl maleimide/hydroxyethylmaleimide copolymers, Isobutylene/MA copolymer, Isobutyl methacrylate/bis-hydroxypropyl dimethicone acrylate copolymers, isopropyl ester of PVM/MA copolymers, lauryl acrylate crosspolymers, lauryl methacrylate/glycol dimethacrylate crosspolymers, MEA sulfite, methacrylic acid/sodium acrylamidomethyl propane sulfonate copolymers, methacryloyl ethyl betaine/acrylates copolymers, octyl acrylamide/acrylates/butylaminoethyl methacrylate copolymers, PEG/PPG-25/25 dimethicone/acrylates copolymers, PEG-8/SMDI copolymers, polyacrylamide, polyacrylate-6, polybeta-alanine/glutaric acid crosspolymers, polybutylene terephthalate, polyester-1, polyethyl acrylate, polyethylene terephthalate, polymethacryloyl ethyl betaine, polypentaerythritol terephthalate, polyperfluoroperhydrophenanthrene, polyquatemium-1, polyquaternium-2, polyquaternium-4, polyquaternium-5, polyquaternium-6, polyquatemium-7, polyquatemium-8, polyquatemium-9, polyquatemium-10, polyquatemium-11, polyquatemium-12, polyquatemium-13, polyquatemium-14, polyquatemium-15, polyquatemium-16, polyquatemium-17, polyquatemium-18, polyquatemium-19, polyquaternium-20, polyquatemium-22, polyquatemium-24, polyquatemium-27, polyquatemium-28, polyquatemium-29, polyquatemium-30, polyquatemium-31, polyquatemium-32, polyquatemium-33 , polyquaternium-34, polyquatemium-35, polyquatemium-36, polyquatemium-39, polyquatemium-45, polyquatemium-46, polyquatemium-47, polyquatemium-48, polyquatemium-49, polyquatemium-50, polyquatemium-55, polyquatemium-56, polysilicone-9, polyurethane-1, polyurethane-6, polyurethane-10, polyvinyl acetate, polyvinyl butyral, polyvinyl caprolactam, polyvinyl formamide, polyvinyl imidazolinium acetate, polyvinyl methyl ether, potassium butyl ester of pvm/ma copolymer, potassium ethyl ester of pvm/ma copolymer, ppg-70 polyglyceryl-10 ether, ppg-12/smdi copolymer, ppg-51/smdi copolymer, ppg-10 sorbitol, pvm/ma copolymer, pvp, pvp/va/itaconic acid copolymer, pvp/vaninyl propionate copolymer, rhizobian gum, rosin acrylate, shellac, sodium butyl ester of pvm/ma copolymer, sodium ethyl ester of pvm/ma copolymer, sodium polyacrylate, sterculia urens gum, terephthalic acid/isophthalic acid/sodium isophthalic acid sulfonate/glycol copolymer, trimethylolpropane triacrylate, trimethylsiloxysilyl-carbamoyl pullulan, VA/crotonates copolymers, VA/crotonates/meth-acryloxybenzophenone-1 copolymers, VA/crotonates/vinyl neodecanoate copolymers, VA/crotonates/vinyl propionate copolymers, VA/DBM copolymers, VA/vinyl butyl benzoate/crotonates copolymers, vinylamine/vinyl alcohol copolymers, vinyl caprolactam/VP/dimethylaminoethyl methacrylate copolymers, VP/acrylate/lauryl methacrylate copolymers, VP/dimethylamino ethyl methacrylate copolymers, VP/DMAPA acrylate copolymers, VP/hexadecene copolymers, VP/VA copolymers, VP/vinyl caprolactam/DMAPA acrylate copolymers, yeast palmitate and styrene/VP copolymers.

Especially preferred are cosmetic products, which are characterized in that the film-forming polymer a3) is selected from the group of anionic polymers, preferably from the group of copolymers of acrylic acid and methacrylic acid.

A particularly preferred anionic acrylate copolymer a3) is synthesized at least from the following monomer units: at least one methacrylic acid unit (1), at least one alkyl (meth)acrylate unit (2) and at least one hydroxyalkyl (meth)acrylate unit (3). This preferred copolymer a3) may be synthesized as contemplated herein from further monomer units. However, in accordance with embodiments as contemplated herein, copolymer a3) is synthesized only from the units (1), (2) and (3), that is, it consists of monomer units derived from these monomers.

The at least one methacrylic acid unit (1) may be a methacrylic acid unit or an acrylic acid unit.

The alkyl group of the alkyl (meth)acrylate unit (2) preferably is a C1-C8 alkyl group, which may be linear or branched. Methyl, ethyl, propyl, isopropyl, 1-butyl, 2-butyl, iso-butyl, t-butyl, linear or branched pentyl, linear or branched hexyl, linear or branched heptyl and linear or branched octyl are examples of alkyl groups. A C1-C5 alkyl group is preferred. In accordance with one embodiment as contemplated herein, two or more alkyl (meth)acrylate units (2) are contained, which differ with respect to the number of carbon atoms in the alkyl group. For example, a C1-C3 alkyl methacrylate unit and a C2-C5 alkyl acrylate unit are contained.

The hydroxyalkyl group of the hydroxyalkyl (meth)acrylate unit (3) may be a hydroxy C1-C10 alkyl group, preferably a hydroxy C2-C5 alkyl group. In a preferred embodiment, the hydroxyalkyl (meth)acrylate unit (3) is hydroxyethyl (meth)acrylate.

The proportion of the (1), (2) and (3) units in the acrylate resin a3) may vary within wide limits. The proportion of unit (1) in the acrylate copolymer preferably is about 2 to about 50% by weight and, more preferably, about 5 to about 30% by weight. The proportion of unit (2) in the acrylate copolymer preferably is about 5 to about 95% by weight, and, more preferably, about 45 to about 90% by weight. The proportion of unit (3) in the acrylate copolymer preferably is about 2 to about 70% by weight and more preferably about 5 to about 30% by weight.

Suitable anionic acrylate copolymers a3) are commercially available under the INCI name of acrylate/hydroxy ester acrylate copolymer. The anionic acrylate copolymer (a), Acudyne® 1000 of the Dow Chemical Company, is most preferred.

A further preferred anionic acrylate copolymer a3) comprises structural units of formula (4), wherein R1 represents a methyl group and R2 represents a methyl group and structural units of formula (4) and wherein R1 represents a hydrogen atom and R2 a butyl group (especially an n-butyl group) and structural units of formula (5), wherein R3 represents a methyl group and R4 a 2-hydroxyethyl group, and structural units of formula (6), wherein R⁷ represents a methyl group and at least one structural unit of formula (7), wherein R⁵ and R⁶, independently of one another, represent a hydrogen atom or a (C₁ to C₆) alkyl group, preferably a hydrogen atom.

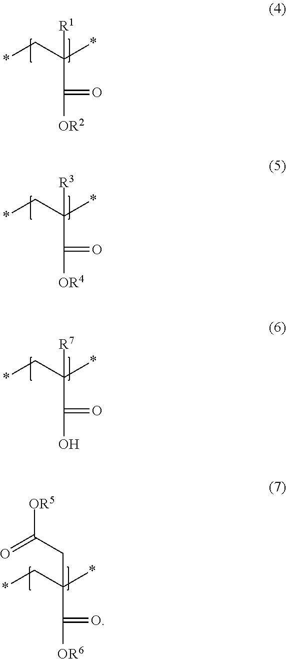

A particular preferred polymer has the INCI name of acrylate/C1-C2 succinate/hydroxy acrylate copolymer. It may be obtained, for example, from Dow under the trade name Acudyne LT-120 (INCI-nomenclature: acrylate/C1-C2 succinate/hydroxyacrylate copolymer).

A second group of polymers a3), preferably used in the cosmetic preparation, are the vinylpyrrolidone homo polymers or copolymers B. Polymers B, the use of which is especially preferred, are, for example:

polyvinylpyrrolidones, such as those sold, for example, under the name of Luviskol® (BASF),

vinylpyrrolicone/vinyl ester copolymers, such as those sold, for example, under the trademark of Luviskol® (BASF). Luviskol® VA 64 and Luviskol® VA 73, each of which is a vinylpyrrolidone/vinyl acetate copolymer, are preferred nonionic polymers.

Because of their cosmetic effect in combination with the copolymers a2), film forming polymers, the use of which is particularly preferred as contemplated herein, are, in particular, the polyvinylpyrrolidones (INCI Name: PVP), as well as the vinylpyrrolidone/vinyl acetate copolymers (INCI name: VP/VA copolymer). Preferred cosmetic products are characterized in that the film-forming polymer a3) is selected from the group of nonionic polymers, preferably from the group of polyvinylpyrrolidones and vinylpyrrolidone/vinyl acetate copolymers, preferably the polyvinylpyrrolidones.

A third group of particularly preferred film-forming polymers a3) comprises amphoteric consolidating polymer C based on

at least one monomer C1, which is selected from acrylic acid, methacrylic acid, alkyl acrylates and alkyl methacrylates, and

at least one amphoteric monomers C2 of formula C2

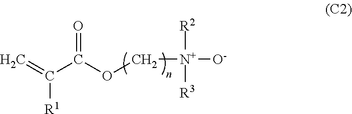

wherein,

R¹ represents H or CH₃,

R² and R³, independently of one another, represent branched C1-10 alkyl and

n represents a whole number from 1 to 20.

In the sense of the present embodiment, only those copolymers C are understood to be additional, amphoteric, consolidating polymers, which are formed from the monomers named and contain, aside from polymer units, which result from the incorporation of the C1 and C monomers named into the copolymer, no more than about 5% by weight and preferably no more than 1% by weight of polymer units, which are attributed to the incorporation of other monomers. Preferably, the copolymers Care synthesized exclusively from the polymer units, which result from the incorporation of the C1and C2 monomers named into the copolymer.

Acrylic acid, methacrylic acid, C1-20 alkyl acrylates and C1-20 alkyl methacrylates are suitable C1 monomers.<0} It is particularly suitable if monomer C1 is selected from acrylic acid, methacrylic acid, methyl acrylate, methyl methacrylate, ethyl acrylate, ethyl methacrylate, propyl acrylate, propyl methacrylate, isopropyl acrylate, isopropyl methacrylate, lauryl acrylate, lauryl methacrylate, acetal acetate, acetal methacrylate, stearyl acrylate and stearyl methacrylate, and particularly preferred if it is selected from acrylic acid, methacrylic acid, methyl acrylate, methyl methacrylate, ethyl acrylate, ethyl methacrylate, lauryl acrylate, lauryl methacrylate, stearyl acrylate and stearyl methacrylate.

Suitable C2 monomers are (meth)acryloyl alkylamine oxides of formula C2, wherein R2 and R3, independently of one another, represent methyl, ethyl, propyl, isopropyl, butyl, isobutyl or t-butyl, methyl being particularly preferred. Suitable C2 monomers furthermore are selected from at least one monomer of the group comprising (meth)acryloyl alkylamine oxides of formula C2, wherein n is a whole number from 1 to 5, preferably a whole number from 1 to 3 and, in particular, 2.

Preferably, C2 monomers are also selected from at least one monomer of the group comprising (meth)acryloyl alkylamine oxides of formula C2, wherein R1 represents CH3. Particularly suitable are the C2 monomers, which are selected from at least one monomer of the group comprising (meth)acryloyl alkylamine oxides of formula C2, wherein R2 and R3, independently of one another, represent methyl, ethyl, propyl, isopropyl, butyl, isobutyl or t-butyl, methyl being particularly preferred, n represents a whole number from 1 to 5, preferably a whole number from 1 to 3 and particularly 2, and R1 represents CH3. It is particularly suitable if the C2 monomer is selected from at least one monomer of the group comprising (meth)acryloyl alkylamine oxides of formula C2, wherein R1, R2 and R3 represent CH3 and n represents 2. Preferred cosmetic products are characterized in that the film-forming polymer a3) is selected from the group comprising the copolymers of methacryloyl ethyl N,N-dimethylamine oxide.

In a particularly preferred embodiment, the inventive cosmetic preparation contains at least one amphoteric, consolidating polymer, which is formed from

at least two C1 monomers, the first monomer being selected from acrylic acid, methacrylic acid, methyl acrylate, methyl methacrylate, ethyl acrylate, ethyl methacrylate, propyl acrylate, propyl methacrylate, isopropyl acrylate and isopropyl methacrylate and the second monomer from stearyl acrylate and stearyl methacrylate, and

as C2 monomer, methacryloyl ethylamine oxide and, in particular, methacryloyl ethyl N,N-dimethylamine oxide (in the formula (A2): R¹═CH³, n=2, R² and R³═CH₃).

These copolymers are also known and obtainable, for example, under the name of Diaformer Z-632 from the Clariant company, the use of Diaformer Z-632 being particularly preferred.

In a suitable embodiment, the inventive material contains at least one amphoteric, consolidating polymer, which is formed from

at least three C1 monomers, wherein the first monomer is selected from acrylic acid, methacrylic acid, methyl acrylate, methyl methacrylate, ethyl acrylate, Ethyl methacrylate, propyl acrylate, propyl methacrylate, propyl acrylate, propyl methacrylate, isopropyl acrylate and isopropyl methacrylate, the second monomer being selected from lauryl acrylate and lauryl methacrylate and the third monomer from stearyl acrylate and stearyl methacrylate and,

as C2 monomer, methacryloyl ethyl amine oxide and, in particular, methacryloyl ethyl N,N-dimethylamine oxide (in Formula (A2):R¹═CH₃, n=2, R² and R³═CH₃).

Corresponding copolymers are also known and obtainable, for example, under the names of Diaformer Z-611, Diaformer Z-612, Diaformer Z-613, Diaformer Z-631, Diaformer Z-633, Diaformer Z-651, Diaformer Z-711 N, Diaformer Z-712N and Diaformer Z-731N from the Clariant company, the use of Diaformer Z-712N and Diaformer Z-651 being preferred.

A fourth group of particularly preferred film-forming polymers a3) comprises copolymers a3), which can be ascribed to the monomers i) N-t-octyl acrylamide, ii) acrylic acid, iii) t-butylamino ethyl methacrylate.

Preferred copolymers a3) preferably comprise at least about 90% by weight, especially at least about 95% by weight and particularly at least about 97% by weight of the monomers N-t-octyl acrylamide, acrylic acid and t-butylamino ethyl methacrylate. Particularly preferred a3) copolymers were obtained exclusively from the monomers, N-t-octyl acrylamide, acrylic acid and t-butyl amino ethyl methacrylate.

It is particularly preferred if a3) copolymers are obtained from the monomers, i) N-t-octyl acrylamide, ii) acrylic acid, iii) t-butyl amino ethyl methacrylate, iv. methyl methacrylate and v) hydroxy-propyl methacrylate.

The above described a3) copolymers are sold, for example, under the name Amphomer® (INCI name: octyl acrylamide/acrylates/butylaminoethyl methacrylate copolymer; CAS Number 70801-07-9) by the National Starch company.

Additional care materials in particular are to be mentioned as further active ingredients or additives.

The material may contain, for example, at least one protein hydrolysate and/or one of its derivatives as care material. Protein hydrolysates are mixtures, which are obtained by acidically, basically or enzymatically catalyzed degradation of. The concept of protein hydrolysates as contemplated herein is understood to include also total hydrolysates, as well as individual amino acids and their derivatives and mixtures of different amino acids. The molecular weight of the protein hydrolysates, which can be used as contemplated herein, is between about 75, the molecular weight of glycine, and about 200,000; preferably it is between about 75 and about 50,000 and, particularly, between about 75 and about 20,000 Dalton.

As care material, the inventive material may furthermore contain at least one vitamin, one provitamin, one vitamin precursor and/or one of their derivatives. Moreover, as contemplated herein, those vitamins, provitamins and vitamin precursors are preferred, which usually are assigned to the groups A, B, C, E, F and H.

Further care materials are panthenol, caffeine, nicotinamide and sorbitol

As care material, the inventive materials may furthermore contain at least one plant extract, but also monosaccharides or oligosaccharides and/or lipids.

The composition of some particularly preferred, inventive, cosmetic preparations is given in the following Tables (unless stated otherwise, the data in weight percent is based on the total weight of the cosmetic material). Reference is made to further preferred embodiments of these particularly preferred compositions, mutatis mutandis, to the inventive cosmetic preparations a) previously described for the cosmetic preparations.

| | Formula 1 | Formula 2 | Formula 3 | Formula 4 | Formula 5 |
|---|---|---|---|---|---|
| Polar solvent a1) | 60 to 98 | 70 to 98 | 80 to 98 | 89 | 92 |
| Copolymer a2) | 0.1 to 20 | 0.5 to 15 | 1.0 to 10 | 7.0 | 4.0 |
| Optional additives | to 100 | to 100 | to 100 | to 100 | to 100 |
| | Formula 6 | Formula 7 | Formula 8 | Formula 9 | Formula 10 |
| Polar solvent a1) | 60 to 98 | 70 to 98 | 80 to 98 | 89 | 92 |
| Copolymer a2) ¹⁾ | 0.1 to 20 | 0.5 to 15 | 1.0 to 10 | 7.0 | 4.0 |
| Optional additives | to 100 | to 100 | to 100 | to 100 | to 100 |
| | Formula 11 | Formula 12 | Formula 13 | Formula 14 | Formula 15 |
| Polar solvent a1) | 60 to 98 | 70 to 98 | 80 to 98 | 89 | 92 |
| Copolymer a2) | 0.1 to 20 | 0.5 to 15 | 1.0 to 10 | 7.0 | 4.0 |
| Polymer a3) | 0.1 to 20 | 0.5 to 15 | 1.0 to 10 | 1.9 | 0.9 |
| Optional Additives | to 100 | to 100 | to 100 | to 100 | to 100 |
| | Formula 16 | Formula 17 | Formula 18 | Formula 19 | Formula 20 |
| Polar solvent a1) | 60 to 98 | 70 to 98 | 80 to 98 | 89 | 92 |
| Copolymer a2) ¹⁾ | 0.1 to 20 | 0.5 to 15 | 1.0 to 10 | 7.0 | 4.0 |
| (Meth)acrylic | 0.1 to 20 | 0.5 to 15 | 1.0 to 10 | 1.9 | 0.9 |
| acid copolymer | | | | | |
| Optional additives | to 100 | to 100 | to 100 | to 100 | to 100 |
| | Formula 21 | Formula 22 | Formula 23 | Formula 24 | Formula 25 |
| Polar Solvent a1) | 60 to 98 | 70 to 98 | 80 to 98 | 89 | 92 |
| Copolymer a2) | 0.1 to 20 | 0.5 to 15 | 1.0 to 10 | 7.0 | 4.0 |
| Vinylpyrrolidone/Vinyl- | 0.1 to 20 | 0.5 to 15 | 1.0 to 10 | 1.9 | 0.9 |
| acetate-Copolymer | | | | | |
| Optional Additives | to 100 | to 100 | to 100 | to 100 | to 100 |
| | Formula 26 | Formula 27 | Formula 28 | Formula 29 | Formula 30 |
| Polar Solvent a1) | 60 to 98 | 70 to 98 | 80 to 98 | 89 | 92 |
| Copolymer a2) ¹⁾ | 0.1 to 20 | 0.5 to 15 | 1.0 to 10 | 7.0 | 4.0 |
| Methacryloylethyl- | 0.1 to 20 | 0.5 to 15 | 1.0 to 10 | 1.9 | 0.9 |
| N,N- | | | | | |
| dimethylaminoxide | | | | | |
| Copolymer | | | | | |
| Optional Additives | to 100 | to 100 | to 100 | to 100 | to 100 |
| | Formula 31 | Formula 32 | Formula 33 | Formula 34 | Formula 35 |
| Polar Solventa1) | 60 to 98 | 70 to 98 | 80 to 98 | 89 | 92 |
| Copolymer a2) | 0.1 to 20 | 0.5 to 15 | 1.0 to 10 | 7.0 | 4.0 |
| N-t-Octyl | 0.1 to 20 | 0.5 to 15 | 1.0 to 10 | 1.9 | 0.9 |
| acrylamide/acrylic | | | | | |
| acid/t-butylamine | | | | | |
| ethyl methacrylate | | | | | |
| copolymer | | | | | |
| Optional additives | to 100 | to 100 | to 100 | to 100 | to 100 |
| | Formula 36 | Formula 37 | Formula 38 | Formula 39 | Formula 40 |
| Ethanol | 60 to 98 | 70 to 98 | 80 to 98 | 89 | 92 |
| Copolymer a2) | 0.1 to 20 | 0.5 to 15 | 1.0 to 10 | 7.0 | 4.0 |
| Optional additives | to 100 | to 100 | to 100 | to 100 | to 100 |
| | Formula 41 | Formula 42 | Formula 43 | Formula 44 | Formula 45 |
| Ethanol | 60 to 98 | 70 to 98 | 80 to 98 | 89 | 92 |
| Copolymer a2) ¹⁾ | 0.1 to 20 | 0.5 to 15 | 1.0 to 10 | 7.0 | 4.0 |
| Optional additives | to 100 | to 100 | to 100 | to 100 | to 100 |
| | Formula 46 | Formula 47 | Formula 48 | Formula 49 | Formula 50 |
| Ethanol | 60 to 98 | 70 to 98 | 80 to 98 | 89 | 92 |
| Copolymer a2) | 0.1 to 20 | 0.5 to 15 | 1.0 to 10 | 7.0 | 4.0 |
| Polymer a3) | 0.1 to 20 | 0.5 to 15 | 1.0 to 10 | 1.9 | 0.9 |
| Optional additives | to 100 | to 100 | to 100 | to 100 | to 100 |
| | Formula 51 | Formula 52 | Formula 53 | Formula 54 | Formula 55 |
| Ethanol | 60 to 98 | 70 to 98 | 80 to 98 | 89 | 92 |
| Copolymer a2) ¹⁾ | 0.1 to 20 | 0.5 to 15 | 1.0 to 10 | 7.0 | 4.0 |
| (Meth)acrylic | 0.1 to 20 | 0.5 to 15 | 1.0 to 10 | 1.9 | 0.9 |
| acid copolymer | | | | | |
| Optional additives | to 100 | to 100 | to 100 | to 100 | to 100 |
| | Formula 56 | Formula 57 | Formula 58 | Formula 59 | Formula 60 |
| Ethanol | 60 to 98 | 70 to 98 | 80 to 98 | 89 | 92 |
| Copolymer a2) | 0.1 to 20 | 0.5 to 15 | 1.0 to 10 | 7.0 | 4.0 |
| Vinylpyrrolidone/vinyl | 0.1 to 20 | 0.5 to 15 | 1.0 to 10 | 1.9 | 0.9 |
| acetate copolymer | | | | | |
| Optional additives | to 100 | to 100 | to 100 | to 100 | to 100 |
| | Formula 61 | Formula 62 | Formula 63 | Formula 64 | Formula 65 |
| Ethanol | 60 to 98 | 70 to 98 | 80 to 98 | 89 | 92 |
| Copolymer a2) ¹⁾ | 0.1 to 20 | 0.5 to 15 | 1.0 to 10 | 7.0 | 4.0 |
| Methacryloyl | 0.1 to 20 | 0.5 to 15 | 1.0 to 10 | 1.9 | 0.9 |
| ethyl N,N- | | | | | |
| dimethylamine | | | | | |
| oxide copolymer | | | | | |
| Optional additives | to 100 | to 100 | to 100 | to 100 | to 100 |
| | Formula 66 | Formula 67 | Formula 68 | Formula 69 | Formula 70 |
| Ethanol | 60 to 98 | 70 to 98 | 80 to 98 | 89 | 92 |
| Copolymer a2) | 0.1 to 20 | 0.5 to 15 | 1.0 to 10 | 7.0 | 4.0 |
| N-t-octyl | 0.1 to 20 | 0.5 to 15 | 1.0 to 10 | 1.9 | 0.9 |
| acrylamide/acrylic | | | | | |
| acid/t-butyl- | | | | | |
| amino ethyl | | | | | |
| methacrylate | | | | | |
| copolymer | | | | | |
| Optional additives | to 100 | to 100 | to 100 | to 100 | to 100 |

¹⁾ Copolymer with INCI name of Styrene/Acrylate Copolymer

Aside from the previously described components a1) to a3), the particularly preferred cosmetic preparations contained only slight amounts of further active ingredients and additives. Cosmetic preparations, characterized in that the weight of the components a1), a2) and, if contained, a3), as a percentage of the total weight of the cosmetic preparation, is at least about 80% by weight, preferably at least about 90% by weight, particularly at least about 93% by weight and more particularly at least about 97% by weight, are especially preferred because they can be produced easily and have a good cosmetic effect. Based on their total weight, particularly preferred cosmetic preparations contain at least about 80% by weight, preferably at least about 90% by weight and particularly at least about 97% by weight of the components a1), a2) and a3).

As stated above, the inventive cosmetic preparations a) are particularly suitable for being applied by employing a device for flash evaporation. A further object of the present application therefore is the use of a cosmetic preparation a), containing, based on its total weight,
a1) about 60 to about 98% by weight of at least one polar solvent;
a2) about 0.1 to about 20% by weight of at least one copolymer from the following monomers

i) styrene

ii) methacrylic acid

iii) as well as optionally further monomers

as process materials in a device for flash evaporation.

The subject matter of the present application moreover is the use of an inventive product for acting upon keratin-containing fibers, especially on human hair, with a cosmetic preparation a) or for the temporary deformation of keratin -containing fibers, especially of human hair.

A method for the temporary deformation of keratin-containing fibers, especially of human hair, for which a device for flash evaporation acts upon the keratin-containing fibers with a cosmetic preparation a) containing, based on its total weight
a1) about 60 to about 98% by weight of at least one polar solvent;
a2) about 0.1 to about 20% by weight of at least one copolymer from the following monomers

i) styrene

ii) methacrylic acid

iii) as well as, optionally, further monomers

is a further subject matter of the present disclosure. The cosmetic preparation a) is converted with the device for flash evaporation preferably into a spray mist, which subsequently acts on the keratin-containing fibers.

In order to achieve a sufficient spraying effect, the cosmetic preparation a) preferably is heated to temperatures above the boiling point of the polar solvent or solvent mixture, contained in the cosmetic preparation a).

If the polar solvent is water or a solvent mixture containing more than 50% by weight of water (based on the total weight of the solvent mixture), the cosmetic preparation preferably is heated to temperatures above about 100° C., preferably to temperatures of about 100° C. to about 240° C. and particularly to temperatures of about 140° C. to about 160° C.

The pressure, achieved by heating the cosmetic preparation a) in the cases in which the polar solvent is water or a solvent mixture containing more than about 50% by weight of water (based on the total weight of the solvent mixture, preferably is between about 1.1 and about 8 bar and particularly between about 1.2 and about 4 bar.

A preferred subject matter of the application is a method for changing the color of keratin-containing fibers, especially human hair, for which the keratin-containing fibers are acted upon by employing a device for flash evaporation with a cosmetic preparation a), which contains, based on its total weight,
a1) about 60 to about 98% by weight of at least one polar solvent;
a2) about 0.1 to about 20% by weight of at least one copolymer from the following monomers

i) styrene

ii) methacrylic acid

iii) as well as, optionally, further monomers,

wherein

from a reservoir, in the interior of which there is a pressure, which corresponds to atmospheric pressure, a partial amount of the cosmetic preparation a), which is in this reservoir, is transferred into a container b1);

wherein the access between the reservoir and the container b1) has a flow-regulating component, such that the flow of the cosmetic preparation a) from the reservoir into the container b1), can be interrupted, is interrupted; subsequently, the cosmetic preparation a) in the container b1), which is closed off from the surroundings, is heated by employing a heater, so that the pressure in the interior of the container b1) rises to values above the atmospheric pressure, preferably to values between about 1.1 and about 8 bar and particularly to values between about 1.2 and about 4 bar;

subsequently, the container b1), which is under a pressure above atmospheric pressure, is opened in such a manner, that at least a partial amount, preferably at least about 50% by weight, more preferably at least about 80% by weight and particularly at least about 90% of the cosmetic preparation in the container b1) escapes from the container b1) into the surroundings with a reduction, at the time that the container is opened, in the pressure existing in the container 1)1).

The pressure on the cosmetic preparation a) is relieved into the surroundings preferably with the formation of a spray mist of the cosmetic preparation a).

The cosmetic preparation a), the pressure on which is relieved as it emerges from the container b1), preferably is applied on keratinic fibers, especially on human hair.

Methods, in the course of which the cosmetic preparation, as the pressure on it is relieved as it leaves the container b1) and before it impacts on the keratinic fibers, is passed through a nozzle, are particularly preferred.

With respect to further preferred embodiments of the inventive uses and of the inventive method, that, which has been said in connection with the inventive cosmetic preparations a) and the device, applies mutatis mutandis to the flash evaporation b).

The inventive materials, uses and methods and some of their preferred embodiments are characterized collectively by the following points:
1. Cosmetic product, comprising

a) a cosmetic preparation containing, based on its total weight,
a1) about 60 to about 98% by weight of at least one polar solvent;

a2) 0.1 to 20% by weight of at least one copolymer of
i) styrene
ii) methacrylic acid
iii) as well as, optionally, further monomers

b) a device for the flash evaporation of the cosmetic preparation a).
2. The cosmetic product according to point 1, characterized in that the device for the flash evaporation comprises a container b1) and a heater b2) and is configured in such a way that

the cosmetic preparation a) can be accommodated in the interior of the container b1),

the interior of the container b1), which is at least partially filled with the cosmetic preparation a), can be closed off,

the cosmetic preparation a) in the closed interior of the container b1) can be heated with the heater b2) while the pressure increases,

the pressure on the heated cosmetic preparation a) in the interior of the container b1) can be relieved by lowering the pressure into the surroundings.
3. The cosmetic pressure according to one of the preceding points, characterized in that the polar solvent a1) constitutes 70 to 98% by weight and preferably 80 to 98% by weight of the total weight of the cosmetic preparation.
4. The cosmetic product according to one of the preceding points, characterized in that the polar solvent a1) has a boiling point (20° C., 1013 mbar) of between about 50 and about 110° C. and preferably of between about 70 and about 105° C.
5. The cosmetic products according to one of the preceding points, characterized in that the polar solvent a1) is selected from the group comprising ethanol, isopropanol and water.
6. The cosmetic products according to one of the preceding points, characterized in that the ratio by weight of water and ethanol to the total weight of the polar solvent a1) preferably is at least about 60% by weight, especially at least about 80% by weight, particularly at least about 90% by weight and, more particularly, at least about 95% by weight.
7. The cosmetic products according to one of the preceding points, characterized in that the polar solvent a1) constitutes more than about 80% by weight and preferably more than about 88% by weight of the total weight of the polar solvent a1).
8. The cosmetic products according to one of the preceding points, characterized in that ethanol constitutes more than about 70% by weight, preferably more than about 80% by weight and particularly more than about 90% by weight of the total weight of the polar solvent a1).
9. The cosmetic products according to one of the preceding points, characterized in that ethanol constitutes no more than about 55% by weight, preferably about 10 to about 55% by weight, particularly about 25 to about 55% by weight and more particularly about 40 to about 55% by weight of the total weight of the cosmetic preparation.
10. The cosmetic products according to one of the preceding points, characterized in that the polar solvent a1) comprises water and ethanol and the ratio by weight of water to ethanol is about 5:1 to about 1:5, preferably about 2:1 to 1:2 and particularly 5:4 to 4:5.
11. The cosmetic product according to one of the preceding points, characterized in that the copolymer a2) constitutes 0.5 to 15% and preferably 1.0 to 10% of the total weight cosmetic preparation a).
12. The cosmetic product according to one of the preceding points, characterized in that the cosmetic preparation a), on the basis of its total weight, contains 0.1 to 20% by weight, preferably 0.5 to 50% by weight and particularly 1.0 to 10% by weight of a film-forming polymer a3).
13. The cosmetic product according to point 12, characterized in that the film-forming polymer a3) is selected from the group of anionic polymers, preferably from the group of copolymers of acrylic acid and methacrylic acid.
14. The cosmetic product according to point 12, characterized in that the film-forming polymer a3) is selected from the group of nonionic polymers, preferably from the group of polyvinylpyrrolidones and vinyl pyrrolidine/vinyl acetate copolymers and preferably from the group of polyvinylpyrrolidones.
15. The cosmetic product according to point 12, characterized in that the film forming polymer a3) is selected from the group of copolymers of methacryloyl ethyl N,N-dimethylamine oxide.
16. The cosmetic product according to point 12, characterized in that the film-forming polymer a3) is selected from the group of copolymers of i) N-t-octyl acrylamide, ii) acrylic acid, iii) t-butyl ethyl methacrylate and iv) optionally further monomers.
17. The cosmetic product according to one of the preceding points, characterized in that the cosmetic preparation a), based on its total weight, consists of at least 80% by weight, preferably of at least 90% by weight and particularly of at least 97% by weight of the components a1) and a2).
18. The cosmetic product according to one of the preceding points, characterized in that the cosmetic preparation a), based on its total weight, consists of at least 80% by weight, preferably of at least 90% by weight and particularly of at least 97% by weight of the components a2) and a3).
19. Use of a cosmetic preparation a) containing, based on its total weight,

a1) 60 to 98% by weight of at least one polar solvent;

a2) 0.1 to 20% by weight of at least one copolymer from the following monomers
i) styrene
ii) methacrylic acid
iii) as well as, optionally, further monomers as process materials in a device for flash evaporation.
20. The use of a product according to one of the points 1 to 18 for impacting on keratin-containing fibers, especially human hair with a cosmetic preparation a).
21. The use according to a product according to one of the points 1 to 18 for temporarily deforming keratin-containing fibers, especially human hair.
22. A method for the temporary deformation of keratin-containing fibers, especially of human hair, for which a device for flash evaporation impacts upon the keratin-containing fibers with a cosmetic preparation a) containing, based on its total weight

a1) 60 to 98% by weight of at least one polar solvent;

a2) 0.1 to 20% by weight of at least one copolymer from the following monomers
i) styrene
ii) methacrylic acid
iii) as well as, optionally, further monomers.
23. The method according to point 22, characterized in that
   from a reservoir, in the interior of which there is a pressure, which corresponds to atmospheric pressure, a partial amount of the cosmetic preparation a), which is in this reservoir, is transferred into a container b1);
   subsequently the access between the reservoir and the container b1) is interrupted a flow-regulating component, such that the flow of the cosmetic preparation a) from the reservoir into the container b1), which can be interrupted, is interrupted
   subsequently, the cosmetic preparation a) in the container b1), which is closed off from the surroundings, is heated by employing a heater, so that the pressure in the interior of the container b1) increases to values above the surrounding pressure, preferably to values between 1.1 and 8 bar and especially to values between 1.2 and 4 bar;
   subsequently, the container b1), which is under a pressure above that of the surroundings, is opened in such a manner, that at least a partial amount, preferably at least 50% by weight, especially of at least 80% by weight and particularly at least 90% of the weight of the cosmetic preparation under pressure in the container b1) escapes from the container into the surroundings with a reduction in the pressure existing in the container b1) at the time that the latter is opened.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic product, comprising:
a) a cosmetic preparation comprising, based on its total weight,
a1) about 60 to about 98% by weight of at least one polar solvent; and
a2) about 0.1 to about 20% by weight of at least one copolymer of,
i) styrene,
ii) methacrylic acid, and
iii) optionally, further monomers; and
b) a device for the flash evaporation of the cosmetic preparation a).

2. The cosmetic product according to claim 1, wherein the device for the flash evaporation comprises a container b1) and a heater b2) and is configured in such a way that
the cosmetic preparation a) can be accommodated in the interior of the container b1),
the interior of the container b1), which is at least partially filled with the cosmetic preparation a), can be closed off;
the cosmetic preparation a) in the closed interior of the container b1) can be heated with the heater b2) with an increase in pressure; and
the pressure on the heated cosmetic preparation a) in the interior of the container b1) can be lowered by being relieved into the surroundings.

3. The cosmetic product according to claim 1, wherein the proportion by weight of the polar solvent a1) is about 70 to about 98% by weight of the total weight of the cosmetic preparation a).

4. The cosmetic product according to claim 1, wherein the polar solvent a1) has a boiling point (20° C., 1013 mbar) of between about 50 and about 110° C.

5. The cosmetic product according to claim 1, wherein the polar solvent a1) is selected from the group of ethanol, isopropanol, water, and combinations thereof.

6. The cosmetic product according to claim 1, wherein the proportion by weight of the copolymer a2) is about 0.5 to about 15% by weight and of the total weight of the cosmetic preparation a).

7. The cosmetic product according to claim 1, wherein based on its total weight, at least about 80% by weight of the cosmetic preparation a) consists of the components a1) and a2).

8. A cosmetic preparation a) comprising, based on its total weight,
a1) about 60 to about 98% by weight of at least one polar solvent; and
a2) about 0.1 to about 20% by weight of at least one copolymer from the following monomers;
i) styrene
ii) methacrylic acid, and
iii) as well as optionally further monomers,
wherein the cosmetic preparation is utilized as process materials in a device for flash evaporation.

9. The cosmetic product according claim 1, wherein the cosmetic product is utilized for impacting on keratin-containing fibers with the cosmetic preparation a).

10. The cosmetic product according claim 1, wherein the cosmetic product is utilized for the temporary deformation of keratin-containing fibers.

11. A method for the temporary deformation of keratin-containing fibers, the method comprising: impacting upon the keratin-containing fibers utilizing a device for flash evaporation with a cosmetic preparation comprising, based on its total weight:
a1) about 60 to about 98% by weight of at least one polar solvent; and
a2) about 0.1 to about 20% by weight of at least one copolymer from the following monomers;
i) styrene
ii) methacrylic acid, and
iii) optionally, further monomers.

12. The method according to claim 11, wherein
from a reservoir, in the interior of which there is a pressure, which corresponds to atmospheric pressure, a partial amount of the cosmetic preparation, which is in this reservoir, is transferred into a container b1);
subsequently the access between the reservoir and the container b1) is interrupted by a flow-regulating component, by means of which such that the flow of the cosmetic preparation a) from the reservoir into the container b1), which can be interrupted, is interrupted;
subsequently, the cosmetic preparation in the container b1), which is closed off from the surroundings, is heated by utilizing a heater, so that the pressure in the interior of the container b1) rises to values above atmospheric pressure;
subsequently, the container b1), which is under a pressure above that of the surroundings, is opened such that at least a partial amount of the weight of the cosmetic preparation under pressure in the container b1) escapes into the surroundings with a reduction in the pressure existing in the container b1) at the time that the latter is opened.

13. The cosmetic product according to claim 1, wherein the proportion by weight of the polar solvent a1) is about 80 to about 98% by weight of the total weight of the cosmetic preparation a).

14. The cosmetic product according to claim 1, wherein the polar solvent a1) has a boiling point (20° C., 1013 mbar) of between about 70 and about 105° C.

15. The cosmetic product according to claim 1, wherein the proportion by weight of the copolymer a2) is about 1.0 to about 10% by weight of the total weight of the cosmetic preparation a).

16. The cosmetic product according to claim 1, wherein the cosmetic preparation a), on the basis of its total weight, comprises about 0.1 to about 20% by weight of a film-forming polymer a3).

17. The cosmetic product according to claim 16, wherein the film-forming polymer a3) is selected from the group of anionic polymers.

18. The cosmetic product according to claim 16, wherein the film-forming polymer a3) is selected from the group of nonionic polymers.

19. The cosmetic product according to claim 16, wherein the film forming polymer a3) is selected from the group of copolymers of methacryloyl ethyl N,N-dimethylamine oxide.

20. The cosmetic product according to claim 16, wherein the film-forming polymer a3) is selected from the group of copolymers of i) N-t-octyl acrylamide, ii) acrylic acid, iii) t-butyl ethyl methacrylate and iv) optionally further monomers.
